SEQUENCE LISTING (10 sequences)

SEQ ID NO: 1 - protein, 917 aa, Homo sapiens
MIAAQLLAYYFTELKDDQVKKIDKYLYAMRLSDETLIDIMTRFRKEMKNGLSRDFNPTATVKMLPTFVRSIPDGSEKGDFIALDLGGSSFRILRVQVNHEKNQNVHMESEVYDTPENIVHGSGSQLFDHVAECLGDFMEKRKIKDKKLPVGFTFSFPCQQSKIDEAILITWTKRFKASGVEGADVVKLLNKAIKKRGDYDANIVAVVNDTVGTMMTCGYDDQHCEVGLIIGTGTNACYMEELRHIDLVEGDEGRMCINTEWGAFGDDGSLEDIRTEFDREIDRGSLNPGKQLFEKMVSGMYLGELVRLILVKMAKEGLLFEGRITPELLTRGKFNTSDVSAIEKNKEGLHNAKEILTRLGVEPSDDDCVSVQHVCTIVSFRSANLVAATLGAILNRLRDNKGTPRLRTTVGVDGSLYKTHPQYSRRFHKTLRRLVPDSDVRFLLSESGSGKGAAMVTAVAYRLAEQHRQIEETLAHFHLTKDMLLEVKKRMRAEMELGLRKQTHNNAVVKMLPSFVRRTPDGTENGDFLALDLGGTNFRVLLVKIRSGKKRTVEMHNKIYAIPIEIMQGTGEELFDHIVSCISDFLDYMGIKGPRMPLGFTFSFPCQQTSLDAGILITWTKGFKATDCVGHDVVTLLRDAIKRREEFDLDVVAVVNDTVGTMMTCAYEEPTCEVGLIVGTGSNACYMEEMKNVEMVEGDQGQMCINMEWGAFGDNGCLDDIRTHYDRLVDEYSLNAGKQRYEKMISGMYLGEIVRNILIDFTKKGFLFRGQISETLKTRGIFETKFLSQIESDRLALLQVRAILQQLGLNSTCDDSILVKTVCGVVSRRAAQLCGAGMAAVVDKIRENRGLDRLNVTVGVDGTLYKLHPHFSRIMHQTVKELSPKCNVSFLLSEDGSGKGAALITAVGVRLRTEASS

SEQ ID NO: 2 - protein, 916 aa, Homo sapiens
MDCEHSLSLPCRGAEAWEIGIDKYLYAMRLSDETLIDIMTRFRKEMKNGLSRDFNPTATVKMLPTFVRSIPDGSEKGDFIALDLGGSSFRILRVQVNHEKNQNVHMESEVYDTPENIVHGSGSQLFDHVAECLGDFMEKRKIKDKKLPVGFTFSFPCQQSKIDEAILITWTKRFKASGVEGADVVKLLNKAIKKRGDYDANIVAVVNDTVGTMMTCGYDDQHCEVGLIIGTGTNACYMEELRHIDLVEGDEGRMCINTEWGAFGDDGSLEDIRTEFDREIDRGSLNPGKQLFEKMVSGMYLGELVRLILVKMAKEGLLFEGRITPELLTRGKFNTSDVSAIEKNKEGLHNAKEILTRLGVEPSDDDCVSVQHVCTIVSFRSANLVAATLGAILNRLRDNKGTPRLRTTVGVDGSLYKTHPQYSRRFHKTLRRLVPDSDVRFLLSESGSGKGAAMVTAVAYRLAEQHRQIEETLAHFHLTKDMLLEVKKRMRAEMELGLRKQTHNNAVVKMLPSFVRRTPDGTENGDFLALDLGGTNFRVLLVKIRSGKKRTVEMHNKIYAIPIEIMQGTGEELFDHIVSCISDFLDYMGIKGPRMPLGFTFSFPCQQTSLDAGILITWTKGFKATDCVGHDVVTLLRDAIKRREEFDLDVVAVVNDTVGTMMTCAYEEPTCEVGLIVGTGSNACYMEEMKNVEMVEGDQGQMCINMEWGAFGDNGCLDDIRTHYDRLVDEYSLNAGKQRYEKMISGMYLGEIVRNILIDFTKKGFLFRGQISETLKTRGIFETKFLSQIESDRLALLQVRAILQQLGLNSTCDDSILVKTVCGVVSRRAAQLCGAGMAAVVDKIRENRGLDRLNVTVGVDGTLYKLHPHFSRIMHQTVKELSPKCNVSFLLSEDGSGKGAALITAVGVRLRTEASS

SEQ ID NO: 3 - protein, 921 aa, Homo sapiens
MGQICQRESATAAEKPKLHLLAESEIDKYLYAMRLSDETLIDIMTRFRKEMKNGLSRDFNPTATVKMLPTFVRSIPDGSEKGDFIALDLGGSSFRILRVQVNHEKNQNVHMESEVYDTPENIVHGSGSQLFDHVAECLGDFMEKRKIKDKKLPVGFTFSFPCQQSKIDEAILITWTKRFKASGVEGADVVKLLNKAIKKRGDYDANIVAVVNDTVGTMMTCGYDDQHCEVGLIIGTGTNACYMEELRHIDLVEGDEGRMCINTEWGAFGDDGSLEDIRTEFDREIDRGSLNPGKQLFEKMVSGMYLGELVRLILVKMAKEGLLFEGRITPELLTRGKFNTSDVSAIEKNKEGLHNAKEILTRLGVEPSDDDCVSVQHVCTIVSFRSANLVAATLGAILNRLRDNKGTPRLRTTVGVDGSLYKTHPQYSRRFHKTLRRLVPDSDVRFLLSESGSGKGAAMVTAVAYRLAEQHRQIEETLAHFHLTKDMLLEVKKRMRAEMELGLRKQTHNNAVVKMLPSFVRRTPDGTENGDFLALDLGGTNFRVLLVKIRSGKKRTVEMHNKIYAIPIEIMQGTGEELFDHIVSCISDFLDYMGIKGPRMPLGFTFSFPCQQTSLDAGILITWTKGFKATDCVGHDVVTLLRDAIKRREEFDLDVVAVVNDTVGTMMTCAYEEPTCEVGLIVGTGSNACYMEEMKNVEMVEGDQGQMCINMEWGAFGDNGCLDDIRTHYDRLVDEYSLNAGKQRYEKMISGMYLGEIVRNILIDFTKKGFLFRGQISETLKTRGIFETKFLSQIESDRLALLQVRAILQQLGLNSTCDDSILVKTVCGVVSRRAAQLCGAGMAAVVDKIRENRGLDRLNVTVGVDGTLYKLHPHFSRIMHQTVKELSPKCNVSFLLSEDGSGKGAALITAVGVRLRTEASS

SEQ ID NO: 4 - protein, 905 aa, Homo sapiens
MAKRALRDFIDKYLYAMRLSDETLIDIMTRFRKEMKNGLSRDFNPTATVKMLPTFVRSIPDGSEKGDFIALDLGGSSFRILRVQVNHEKNQNVHMESEVYDTPENIVHGSGSQLFDHVAECLGDFMEKRKIKDKKLPVGFTFSFPCQQSKIDEAILITWTKRFKASGVEGADVVKLLNKAIKKRGDYDANIVAVVNDTVGTMMTCGYDDQHCEVGLIIGTGTNACYMEELRHIDLVEGDEGRMCINTEWGAFGDDGSLEDIRTEFDREIDRGSLNPGKQLFEKMVSGMYLGELVRLILVKMAKEGLLFEGRITPELLTRGKFNTSDVSAIEKNKEGLHNAKEILTRLGVEPSDDDCVSVQHVCTIVSFRSANLVAATLGAILNRLRDNKGTPRLRTTVGVDGSLYKTHPQYSRRFHKTLRRLVPDSDVRFLLSESGSGKGAAMVTAVAYRLAEQHRQIEETLAHFHLTKDMLLEVKKRMRAEMELGLRKQTHNNAVVKMLPSFVRRTPDGTENGDFLALDLGGTNFRVLLVKIRSGKKRTVEMHNKIYAIPIEIMQGTGEELFDHIVSCISDFLDYMGIKGPRMPLGFTFSFPCQQTSLDAGILITWTKGFKATDCVGHDVVTLLRDAIKRREEFDLDVVAVVNDTVGTMMTCAYEEPTCEVGLIVGTGSNACYMEEMKNVEMVEGDQGQMCINMEWGAFGDNGCLDDIRTHYDRLVDEYSLNAGKQRYEKMISGMYLGEIVRNILIDFTKKGFLFRGQISETLKTRGIFETKFLSQIESDRLALLQVRAILQQLGLNSTCDDSILVKTVCGVVSRRAAQLCGAGMAAVVDKIRENRGLDRLNVTVGVDGTLYKLHPHFSRIMHQTVKELSPKCNVSFLLSEDGSGKGAALITAVGVRLRTEASS

SEQ ID NO: 5 - protein, 917 aa, Homo sapiens
MIASHLLAYFFTELNHDQVQKVDQYLYHMRLSDETLLEISKRFRKEMEKGLGATTHPTAAVKMLPTFVRSTPDGTEHGEFLALDLGGTNFRVLWVKVTDNGLQKVEMENQIYAIPEDIMRGSGTQLFDHIAECLANFMDKLQIKDKKLPLGFTFSFPCHQTKLDESFLVSWTKGFKSSGVEGRDVVALIRKAIQRRGDFDIDIVAVVNDTVGTMMTCGYDDHNCEIGLIVGTGSNACYMEEMRHIDMVEGDEGRMCINMEWGAFGDDGSLNDIRTEFDQEIDMGSLNPGKQLFEKMISGMYMGELVRLILVKMAKEELLFGGKLSPELLNTGRFETKDISDIEGEKDGIRKAREVLMRLGLDPTQEDCVATHRICQIVSTRSASLCAATLAAVLQRIKENKGEERLRSTIGVDGSVYKKHPHFAKRLHKTVRRLVPGCDVRFLRSEDGSGKGAAMVTAVAYRLADQHRARQKTLEHLQLSHDQLLEVKRRMKVEMERGLSKETHASAPVKMLPTYVCATPDGTEKGDFLALDLGGTNFRVLLVRVRNGKWGGVEMHNKIYAIPQEVMHGTGDELFDHIVQCIADFLEYMGMKGVSLPLGFTFSFPCQQNSLDESILLKWTKGFKASGCEGEDVVTLLKEAIHRREEFDLDVVAVVNDTVGTMMTCGFEDPHCEVGLIVGTGSNACYMEEMRNVELVEGEEGRMCVNMEWGAFGDNGCLDDFRTEFDVAVDELSLNPGKQRFEKMISGMYLGEIVRNILIDFTKRGLLFRGRISERLKTRGIFETKFLSQIESDCLALLQVRAILQHLGLESTCDDSIIVKEVCTVVARRAAQLCGAGMAAVVDRIRENRGLDALKVTVGVDGTLYKLHPHFAKVMHETVKDLAPKCDVSFLQSEDGSGKGAALITAVACRIREAGQR

SEQ ID NO: 6 - protein, 25 aa, Homo sapiens
MIAAQLLAYYFTELKDDQVKKIDKY

SEQ ID NO: 7 - protein, 25 aa, Homo sapiens
MDCEHSLSLPCRGAEAWEIGIDKYL

SEQ ID NO: 8 - protein, 25 aa, Homo sapiens
MGQICQRESATAAEKPKLHLLAESE

SEQ ID NO: 9 - protein, 25 aa, Homo sapiens
MAKRALRDFIDKYLYAMRLSDETLI

SEQ ID NO: 10 - protein, 25 aa, Homo sapiens
MIASHLLAYFFTELNHDQVQKVDQY

(19) United States
(12) Patent Application Publication
(10) Pub. No.: US 2025/0295616 A1
(43) Pub. Date: Sep. 25, 2025

(54) A SYSTEM AND METHOD FOR COMPARTMENTALIZED INGREDIENTS FOR A LIQUID PHARMACEUTICAL FORMULATION

(71) Applicant: KODISCOVERY, LLC, Baltimore, MD (US)

(72) Inventor: Young Hee KO, Baltimore, MD (US)

(21) Appl. No.: 18/862,904

(22) PCT Filed: May 2, 2023
(86) PCT No.: PCT/US23/66520

§ 371 Date: Nov. 4, 2024

Related U.S. Application Data
(60) Provisional application No. 63/337,573, filed on May 2, 2022.

(51) Int. Cl. A61K 31/19; A61K 31/122; A61K 31/366; A61K 31/375; A61K 31/385; A61K 31/7004; A61K 31/7084; A61K 31/7088; A61K 36/185; A61K 36/22; A61K 36/815; A61K 36/889; A61K 36/899; A61K 38/10; A61K 45/06; A61K 47/10; A61K 47/24; A61K 47/26; C07K 7/08

(52) U.S. Cl. CPC A61K 31/19; A61K 31/122; A61K 31/366; A61K 31/375; A61K 31/385; A61K 31/7004; A61K 31/7084; A61K 31/7088; A61K 36/185; A61K 36/22; A61K 36/815; A61K 36/889; A61K 36/899; A61K 38/10; A61K 45/06; A61K 47/10; A61K 47/24; A61K 47/26; C07K 7/08

(57) ABSTRACT

A system of compartmentalized ingredients for a liquid pharmaceutical formulation having a first vessel including a cellular energy inhibitor according to formula I in a first liquid carrier, wherein R is selected from one of OR′, N(R″)2, C(O)R″, C1-C6 alkyl, C6-C12 aryl, C1-C6 heteroalkyl, C6-C12 heteroaryl, H, or an alkali metal, where R′ is selected from one Br R of H, an alkali metal, C1-C6 alkyl, C6-C12 aryl or C(O)R′″, where R¹ is selected from one of SH, C1-C6 alkyl, or C6-C12 aryl, and where R′″ is selected from one of H, C1-C20 alkyl or C6-C12 aryl, and a second vessel including an excipient in a second liquid carrier that is chemically reactive with the cellular energy inhibitor, wherein admixing the first liquid carrier with the CA second liquid carrier creates a finished liquid dosage form.

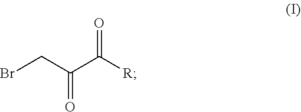

### CROSS-REFERENCE TO RELATED APPLICATIONS

This application claims the benefit of U.S. Provisional Patent Application No. 63/337,573, filed on May 2, 2022, which is incorporated herein by reference in its entirety.

### BACKGROUND

Pharmaceutical formulations have a wide variety of physical forms and compositional formulations, depending on the active agent in the formulation, the route of administration, etc. For example, a solid pharmaceutical formulation includes an active agent dispersed in a solid pharmaceutical carrier. Similarly, a liquid pharmaceutical formulation includes an active agent dispersed in a liquid pharmaceutical carrier. Additional additives can vary depending on whether the dosage form is a liquid or solid, for example. General categories include diluents, disintegrants, binding agents, adhesives, wetting agents, lubricants, glidants, dyes, flavoring agents, to name a few.

### BRIEF DESCRIPTION OF THE DRAWINGS

FIG. 1 illustrates a compartmentalized liquid ingredient pharmaceutical system in accordance with an example embodiment;

FIG. 2A illustrates a compartmentalized liquid ingredient pharmaceutical system in accordance with an example embodiment;

FIG. 2B illustrates a compartmentalized liquid ingredient pharmaceutical system in accordance with an example embodiment;

FIG. 3 illustrates a compartmentalized liquid ingredient pharmaceutical system in accordance with an example embodiment;

FIG. 4 illustrates a compartmentalized liquid ingredient pharmaceutical system in accordance with an example embodiment;

FIG. 5 illustrates a compartmentalized liquid ingredient pharmaceutical system in accordance with an example embodiment; and

FIG. 6 illustrates a compartmentalized liquid ingredient pharmaceutical system in accordance with an example embodiment.

### DESCRIPTION OF EMBODIMENTS

Although the following detailed description contains many specifics for the purpose of illustration, a person of ordinary skill in the art will appreciate that many variations and alterations to the following details can be made and are considered included herein. Accordingly, the following embodiments are set forth without any loss of generality to, and without imposing limitations upon, any claims set forth. It is also to be understood that the terminology used herein is for describing particular embodiments only, and is not intended to be limiting. Unless defined otherwise, all technical and scientific terms used herein have the same meaning as commonly understood by one of ordinary skill in the art to which this disclosure belongs. Also, the same reference numerals in appearing in different drawings represent the same element. Numbers provided in flow charts and processes are provided for clarity in illustrating steps and operations and do not necessarily indicate a particular order or sequence.

Furthermore, the described features, structures, or characteristics can be combined in any suitable manner in one or more embodiments. In the following description, numerous specific details are provided, such as examples of layouts, distances, network examples, etc., to provide a thorough understanding of various embodiments. One skilled in the relevant art will recognize, however, that such detailed embodiments do not limit the overall concepts articulated herein, but are merely representative thereof. One skilled in the relevant art will also recognize that the technology can be practiced without one or more of the specific details, or with other methods, components, layouts, etc. In other instances, well-known structures, materials, or operations may not be shown or described in detail to avoid obscuring aspects of the disclosure.

In this application, “comprises,” “comprising,” “containing” and “having” and the like can have the meaning ascribed to them in U.S. Patent law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the compositions nature or characteristics would be permissible if present under the “consisting essentially of” language, even though not expressly recited in a list of items following such terminology. When using an open-ended term in this written description, like “comprising” or “including,” it is understood that direct support should be afforded also to “consisting essentially of” language as well as “consisting of” language as if stated explicitly and vice versa.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a given term, metric, value, range endpoint, or the like. The degree of flexibility for a particular variable can be readily determined by one skilled in the art. However, unless otherwise expressed, the term “about” generally provides flexibility of less than 0.01%. It is to be understood that, even when the term “about” is used in the present specification in connection with a specific numerical value, support for the exact numerical value recited apart from the “about” terminology is also provided.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 1.5, 2, 2.3, 3, 3.8, 4, 4.6, 5, and 5.1 individually. This same principle applies to ranges reciting only one numerical value as a minimum or a maximum. Furthermore, such an interpretation should apply regardless of the breadth of the range or the characteristics being described.

Reference throughout this specification to “an example” means that a particular feature, structure, or characteristic described in connection with the example is included in at least one embodiment. Thus, appearances of phrases including “an example” or “an embodiment” in various places throughout this specification are not necessarily all referring to the same example or embodiment.

The terms “first,” “second,” “third,” “fourth,” and the like in the description and in the claims, if any, are used for distinguishing between similar elements and not necessarily for describing a particular sequential or chronological order. It is to be understood that the terms so used are interchangeable under appropriate circumstances such that the embodiments described herein are, for example, capable of operation in sequences other than those illustrated or otherwise described herein. Similarly, if a method is described herein as comprising a series of steps, the order of such steps as presented herein is not necessarily the only order in which such steps may be performed, and certain of the stated steps may possibly be omitted and/or certain other steps not described herein may possibly be added to the method.

The formulations of the present invention may include a pharmaceutically acceptable carrier and other ingredients as dictated by the particular needs of the specific dosage formulation. Such ingredients are well known to those skilled in the art. See for example, Gennaro, A. Remington: The Science and Practice of Pharmacy 19^{th} ed. (1995), which is incorporated by reference in its entirety.

As used herein, “administration,” and “administering” refer to the manner in which a composition is presented to a subject. Administration can be accomplished by various art-known routes such as enteral, parenteral, transdermal, and the like, including combinations thereof in some cases. Thus, an enteral administration can be achieved by drinking, swallowing, chewing, sucking of an oral dosage form comprising an active agent or other compound to be delivered. Parenteral administration can be achieved by injecting a drug composition intravenously, intra-arterially, intramuscularly, intrathecally, subcutaneously, etc. Transdermal administration can be accomplished by applying, pasting, rolling, attaching, pouring, pressing, rubbing, etc., of a transdermal preparation onto a skin surface. These and additional methods of administration are well-known in the art.

As used herein, “subject” refers to a mammal that may benefit from the administration of a drug composition or method of this invention. Examples of subjects include humans, and other animals such as horses, pigs, cattle, sheep, goats, dogs (felines), cats (canines), rabbits, rodents, primates, and aquatic mammals. In one embodiment, subject can refer to a human.

As used herein, “drug,” “active agent,” “bioactive agent,” “pharmaceutically active agent,” “therapeutically active agent” “pharmaceutical,” and “active pharmaceutical ingredient (API),” may be used interchangeably to refer to an agent or substance that has measurable specified or selected physiologic activity when administered to a subject in a significant or effective amount. It is to be understood that the term “drug” is expressly encompassed by the present definition as many drugs and prodrugs are known to have specific physiologic activities. These terms of art are well-known in the pharmaceutical and medicinal arts. Further, when these terms are used, or when a particular active agent is specifically identified by name or category, it is understood that such recitation is intended to include the active agent per se, as well as pharmaceutically acceptable salts, or compounds significantly related thereto, including without limitation, prodrugs, active metabolites, isomers, and the like. The terms “cellular energy inhibitor,” “glycolysis inhibitor,” “mitochondrial inhibitor,” and the like, are considered to be active agents.

As used herein, the terms “inhibit,” “inhibiting,” or any other derivative thereof refers to the process of holding back, suppressing or restraining so as to block, prevent, limit, or decrease a rate of action or function. The use of the term is not to be misconstrued to be only of absolute prevention but can be a referent to any minute incremental step of limiting or reducing a function through the full and absolute prevention of the function.

As used herein, “cellular energy inhibitor” refers to a compound that inhibits ATP production in a cell. In some examples, a cellular energy inhibitor can inhibit glycolysis, oxidative phosphorylation, or both glycolysis and oxidative phosphorylation in a cell.

As used herein, “glycolysis inhibitor” refers to a compound that inhibits, reduces, or stops, glycolysis in a cell.

As used herein, “mitochondria inhibitor” refers to a compound that inhibits, reduces, or stops mitochondrial production of ATP in a cell.

As used herein, the terms “dosage form,”, “formulation” and “composition” are used interchangeably and refer to a mixture of two or more compounds, elements, or molecules. In some examples, the terms “dosage form,” “formulation,” and “composition” may be used to refer to a mixture of one or more active agents with a carrier and/or other excipient.

As used herein, “carrier” or “pharmaceutically acceptable carrier” refers to a substance with which a drug may be combined to achieve a specific dosage formulation for delivery to a subject. In some examples, a carrier may or may not enhance drug delivery. As a general principle, carriers do not react with the drug in a manner that substantially degrades or otherwise adversely affects the drug, except that some carriers may react with a drug to prevent it from exerting a therapeutic effect until the drug is released from the carrier. Further, the carrier, or at least a portion thereof must be physiologically suitable for administration into a subject along with the drug.

The term “excipient” herein includes any substance used, for example, as a carrier for an active agent in a liquid formulation, any substance added to the active agent and/or a solid formulation to, for example, improve its handling properties, permit the resulting composition to be formed into an appropriate storage form, facilitating disintegration in a liquid, or the like. Excipients can include, by way of illustration and not by limitation, diluents, disintegrants, binding agents, adhesives, wetting agents, lubricants, glidants, dyes, and any other substance other than the active ingredient conventionally used in the preparation of a liquid or solid formulation.

The terms “reaction” and “react” include any form of chemical change that occurs to a formulation ingredient as a result of contact with another formulation ingredient, including reactions that activate one or more molecules or ingredients (e.g., the change of a precursor to an active agent into the active agent), reactions that degrade at least one ingredient, or the like.

As used herein, “admixed” means that at least two components of the composition can be partially or fully mixed, dispersed, suspended, dissolved, or emulsified in one another. In some cases, at least a portion of the drug may be admixed in at least one carrier substance.

An initial overview of embodiments is provided below, and specific embodiments are then described in further detail. This initial summary is intended to aid readers in understanding the disclosure more quickly and is not intended to identify key or essential technological features, nor is it intended to limit the scope of the claimed subject matter.

Many liquid formulations can include, among other things, an active agent dispersed in a liquid carrier such as, for example, a pharmaceutical carrier. Liquid formulations, however, suffer from several disadvantages. For example, reactive molecules tend to react more readily in a liquid medium. As such, many active agents have reduced potency/efficacy following exposure to a reactive molecule in a liquid medium for a prolonged period of time. Additionally, a volume of a liquid pharmaceutical ingredient can generally be measured more accurately compared to a quantity of a dry pharmaceutical ingredient.

The present disclosure provides a compartmentalized system and method that separates and maintains pharmaceutical formulation ingredients in discrete liquid forms that are isolated from one another. For example, formulations having ingredients that are reactive with one another can be isolated and admixed together as needed to create a finished pharmaceutical product, thus reducing the degradation of the ingredients. As another example, the compartmentalized system maintains an active pharmaceutical ingredient (API) in a convenient liquid form for ready use that is in a separate vessel from ingredients that would react with the API. Isolating the reactive ingredient(s) from the API prolongs the potency of the API, thus allowing the ingredients for the pharmaceutical formulation to be maintained in a convenient liquid form for longer periods of time.

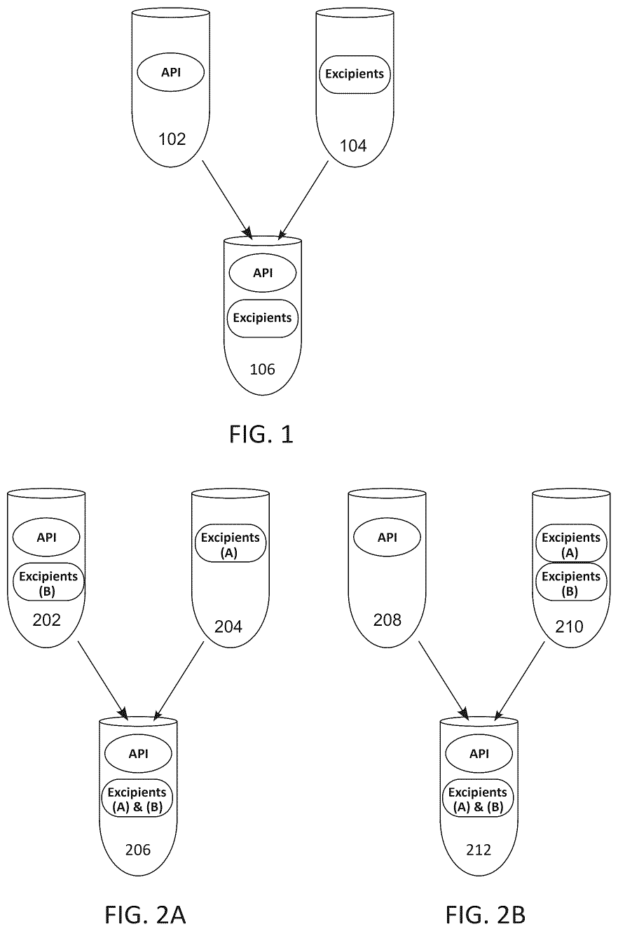

In one example, as is shown in FIG. 1, a compartmentalized system can include a first vessel 102 including an active pharmaceutical ingredient (API) in a first liquid carrier and a second vessel 104 including an excipient in a second liquid carrier that is chemically reactive with the API. When the first liquid carrier is admixed with the second liquid carrier, the API and the excipient form a finished pharmaceutical product in a third vessel 106. Such a system allows formulation ingredients to be mixed as needed, which not only extends the potency of the API, but reduces API and excipient waste. Due to the reactivity between the API and the excipient, the API generally has a lower chemical stability in the finished liquid dosage form compared to the API prior to admixing.

In many cases a given formulation can include additional excipients that can be included in ether the first liquid carrier or the second liquid carrier, depending, in some cases, on the reactivity of the additional excipient(s) with either the API or the excipient in the second liquid carrier. In one example, FIG. 2A shows a first vessel 202 including an API in a first liquid carrier and a second vessel 204 including an excipient that is chemically reactive with the API in a second liquid carrier. The system additionally includes an additional excipient that is not reactive or that is less reactive with the API as compared to the excipient in the first liquid carrier. When the first liquid carrier is admixed with the second liquid carrier, the API, the additional excipient, and the excipient form a finished pharmaceutical product in a third vessel 106. Such a system allows formulation ingredients to be mixed as needed, which not only extends the potency of the API, but reduces API and excipient waste. Due to the reactivity between the API and the excipient, the API generally has a lower chemical stability in the finished liquid dosage form compared to the API prior to admixing.

In another example, FIG. 2B shows a first vessel 208 including an API in a first liquid carrier and a second vessel 210 including an excipient and an additional excipient in a second liquid carrier. In some cases, either one or both of the excipients in the second vessel 210 is/are chemically reactive with the API. In other cases, either one or both of the excipients in the second vessel 210 is not chemically reactive with the API. In other cases, the additional excipient is not chemically reactive with the API. When the first liquid carrier is admixed with the second liquid carrier, the API, the additional excipient, and the excipient form a finished pharmaceutical product in a third vessel 212. Such a system allows formulation ingredients to be mixed as needed, which not only extends the potency of the API, but reduces API and excipient waste. In cases where there is chemical reactivity between the API and the excipient, the API generally has a lower chemical stability in the finished liquid dosage form compared to the API prior to admixing.

The ingredients in the various liquid vessels can be prepared by any technique known in the pharmaceutical arts. In one convenient example, the first liquid formulation, the second liquid formulation, or both, can be made up by introducing a dissolvable tablet (or capsule, fizzy tablet, or the like) into the appropriate carrier to form the isolated ingredients that can be combined with other ingredients to make up the finished pharmaceutical product. As such, when the tabled is introduced into either liquid carrier, the dissolution of the tablet releases the API and the excipient(s) into the appropriate liquid carrier to form the isolated components of the finished pharmaceutical formulation.

In one specific example, the API can be a molecule according to Formula I:

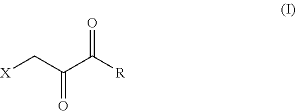

Various specific molecules are contemplated, wherein, for example, X can be, without limitation, a nitro, an imidazole, a halide, sulfonate, a carboxylate, an alkoxide, amine oxide, or the like. Additionally, R can be, without limitation, OR′, N(R″)₂, C(O)R′″, C1-C6 alkyl, C6-C12 aryl, C1-C6 heteroalkyl, a C6-C12 heteroaryl, H, an alkali metal or the like, where R′ represents H, alkali metal, C1-C6 alkyl, C6-C12 aryl or C(O)R′″, R″ represents H, C1-C6 alkyl, or C6-C12 aryl, and R′″ represents H, C1-C20 alkyl or C6-C12 aryl.

In one example, R of formula (I) can be OH and X of formula (I) can be a nitro, an imidazole, a halide, a sulfonate, a carboxylate, an alkoxide, an amine oxide, or the like. Additionally, X can be a halide, such as, for example, fluoride, bromide, chloride, iodide, or the like. In one example, X can be a sulfonate, such as, for example, a triflate, a mesylate, a tosylate, or the like. In another example, X can be amine oxide. In still another example, the amine oxide can be dimethylamine oxide.

In another example, the API can be a 3-halopyruvate, such as, for example, 3-fluoropyruvate, 3-chloropyruvate, 3-bromopyruvate, 3-iodopyruvate, or a combination thereof. A general structure showing a halide in the 3-position is shown in formula II.

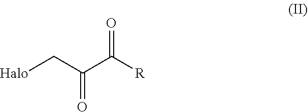

In a further nonlimiting example, the API can have bromine in the 3-position, as shown in formula III.

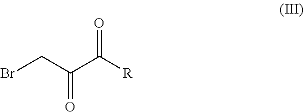

In one further nonlimiting example, the API can be 3-bromopyruvic acid, as shown in formula IV.

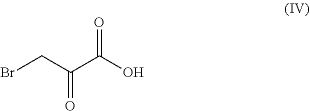

In another nonlimiting example, the API can be 3-bromopyrate, as shown in formula V.

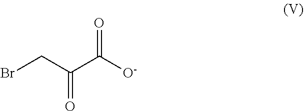

It is noted that both 3-bromopyruvic acid and 3-bromopyrate can be referred to herein using the abbreviation 3-BP. One skilled in the art can readily distinguish between the moieties using this abbreviation depending on the particular context.

In some examples, the API can be formulated in a composition with at least one sugar, which can stabilize the API by substantially preventing the API from hydrolyzing. In some examples, a composition can include 3-BP, as a cellular energy inhibitor, for example, and at least one sugar, at least two sugars, at least three sugars, and the like. In one example, a sugar can include a monosaccharide, a disaccharide, an oligosaccharide, or a combination thereof. Nonlimiting examples of monosaccharides can include glucose, fructose, galactose, and the like. Nonlimiting examples of disaccharides can include sucrose, lactose, maltose, and the like. It is noted that, for the purposes of the present disclosure, the term “sugar” can also include oligosaccharides, polysaccharides, polyols, polyalcohols, and similar molecules that function to stabilize 3-BP.

A sugar can include a 3-carbon sugar, a 4-carbon sugar, a 5-carbon sugar, a 6-carbon sugar, a 7-carbon sugar, and the like, including combinations thereof. In one aspect, the sugar can be a 3-carbon sugar, a 4-carbon sugar, a 5-carbon sugar, a 6-carbon sugar, a 7-carbon sugar, and the like, including combinations thereof, provided the sugar is not involved in energy metabolism to the extent that it generates energy (i.e., a nonmetabolizable sugar).

In one example, the sugar can be gluconic acid. In another example, the sugar can be glucuronic acid. At least one of the sugars can be a five-carbon sugar. In one example, at least two of the sugars can be five-carbon sugars. The five-carbon sugars can be independently selected from mannitol, erythritol, isomalt, lactitol, maltitol, sorbitol, xylitol, dulcitol, ribitol, inositol, or the like, including combinations thereof. In one example, at least one of the sugars can be glycerol. In another example, the sugars can be glycerol, inositol, and sorbitol. Other nonlimiting example of sugars can include ethylene glycol, threitol, arabitol, galactitol, fucitol, iditol, volemitol, maltotriitol, maltotetraitol, and polyglycitol, including combinations thereof. In one example, the sugars can include glycerol, inositol, sorbitol, mannitol or any combination thereof. In another example, the sugars can include glycerol, inositol, sorbitol, or any combination thereof. In yet another example, the inositol can be myo-inositol. In other examples, the sugar can be a polyalcohol.

The sugars described herein can be any isomeric form. In one example, the compositions described herein can include the less biologically active form of the sugar as compared to its isomer. In one case, the less biologically active sugar can be the L-enantiomer sugar. However, if the D-enantiomer sugar is found to be less biologically active as compared to its L form, then the D form can be used. In one example, such sugars can function as a glycolytic inhibitor.

In one example, a composition can include one or more sugars in a range from about 0.5 wt % to about 50.0 wt % or from about 1.0 wt % to about 25.5 wt %. In yet another example, a composition can include one or more sugars in a range from about 0.2 wt % to about 75.0 wt % or from about 0.5 wt % to about 50.0 wt %. In a further example, a composition can include one or more sugars in a range from about 0.1 wt % to about 25.0 wt %, from about 0.2 wt % to about 10.0 wt %.

In some examples, the composition can include glycerol in a range from about 0.1 wt % to about 5.0 wt % or from about 0.1 wt % to about 3.0 wt %. In other examples, the composition can include inositol in a range from about 0.1 wt % to about 10 wt %, from about 0.1 wt % to about 6 wt %. In further examples, the composition can include sorbitol in a range from about 0.1 wt % to about 40.0 wt % or from about 0.1 wt % to about 30 wt %. In yet further examples, the composition can include mannitol in a range from about 0.1 wt % to about 30 wt % or from about 0.1 wt % to about 10 wt %. Additionally, each of the sugars may be added in a volume up to a maximum solubility of the sugar in the formulation or composition. It is additionally noted that the above wt % s of ingredients are without water or other liquid carrier.

In some examples, a 3-BP composition can include a biological buffer that is present in an amount sufficient to at least partially deacidify the cellular energy inhibitor and neutralize metabolic by-products of the cellular energy inhibitor. Nonlimiting examples of biological buffers can include a citrate buffer, a phosphate buffer, an acetate buffer, and the like, including combinations thereof. In one specific example, the biological buffer can be a citrate buffer, such as, without limitation, sodium citrate. In another specific example, the biological buffer can be a phosphate buffer, such as, without limitation, sodium phosphate. In one specific example, the biological buffer can be an acetate buffer, such as, without limitation, sodium acetate. In yet other examples, the biological buffer can include at least two biological buffers, such as, without limitation, a citrate buffer and an acetate buffer, a citrate buffer and a phosphate buffer, an acetate buffer and a phosphate buffer, or a citrate buffer, a phosphate buffer, and an acetate buffer.

In some examples, the composition can include the biological buffer in a range of from about 0.1 wt % to about 15 wt % or from about 2.0 wt % to about 8.0 wt %. Additionally, the biological buffer can maintain a physiological pH of 4.0 to 8.5. In one embodiment, the biological buffer can maintain a physiological pH of 5.5 to 8.0. In another embodiment, the biological buffer can maintain a physiological pH of 6.8 to 7.8. In still another embodiment, the biological buffer can maintain a physiological pH of 7.3 to 7.6. It is additionally noted that the above wt % s of ingredients are without water or other liquid carrier.

In one example, a compartmentalized system and method is provided that separates and maintains 3-BP and an excipient into discrete liquid forms that are isolated from one another. For example, a biological buffer can react with 3-BP, and as such, the biological buffer can be isolated from the 3-BP until they are admixed together to form a finished 3-BP product or a component of a finished 3-BP product. Such isolation can reduce the degradation of the 3-BP while maintaining it in a liquid form that is ready to use when a finished 3-BP product is needed. Isolating the aforementioned ingredients from one another prolongs the potency of the 3-BP, thus allowing the ingredients for the to be maintained in a convenient liquid form for longer periods of time.

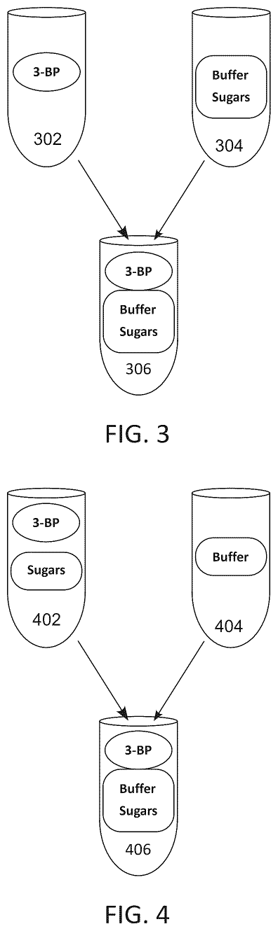

In one example, as is shown in FIG. 3, a compartmentalized system can include a first vessel 302 including 3-BP in a first liquid carrier and a second vessel 304 including a biological buffer in a second liquid carrier. As noted in the second vessel 304, in some cases one or more sugars can be included in the second carrier with the biological buffer. When the first liquid carrier is admixed with the second liquid carrier, the 3-BP and the biological buffer (and, in some cases, the sugar(s)) form a finished 3-BP product in a third vessel 306. Such a system allows formulation ingredients to be mixed as needed, which not only extends the potency of the 3-BP but reduces 3-BP and biological buffer waste. Due to the reactivity between the 3-BP and the biological buffer, the 3-BP generally has a lower chemical stability in the finished liquid dosage form compared to the 3-BP prior to admixing.

In another example, FIG. 4 shows a first vessel 402 including 3-BP in a first liquid carrier and a second vessel 404 including a biological buffer in a second liquid carrier that is chemically reactive with the 3-BP. The system additionally includes one or more sugars in the first liquid carrier with the 3-BP. When the first liquid carrier is admixed with the second liquid carrier, the 3-BP, the sugar(s), and the biological buffer form a finished 3-BP product in a third vessel 406. Such a system allows formulation ingredients to be mixed as needed, which not only extends the potency of the 3-BP, but reduces 3-BP waste. Due to the reactivity between the 3-BP and the excipient, the 3-BP generally has a lower chemical stability in the finished liquid dosage form compared to the API prior to admixing.

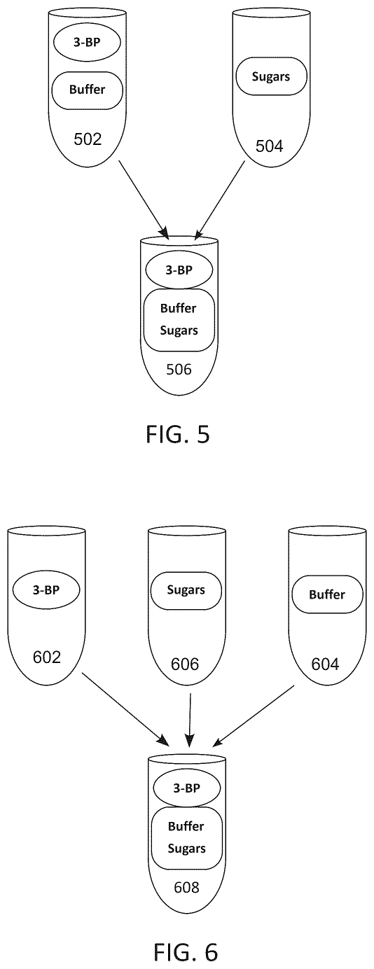

In another example, FIG. 5 shows a first vessel 502 including 3-BP and a biological buffer in a first liquid carrier and a second vessel 504 including one or more sugars in a second liquid carrier. When the first liquid carrier is admixed with the second liquid carrier, the 3-BP, the sugar(s), and the biological buffer form a finished 3-BP product in a third vessel 506. Such a system allows formulation ingredients to be mixed as needed, which not only extends the potency of the 3-BP, but reduces 3-BP waste. Due to the reactivity between the 3-BP and the excipient, the 3-BP generally has a lower chemical stability in the finished liquid dosage form compared to the API prior to admixing.

In yet another example, FIG. 6 shows a first vessel 602 including 3-BP in a first liquid carrier, a second vessel 604 including a biological buffer, and a third vessel 606 including one or more sugars in a third liquid carrier. Other excipients can be added to one or more of the first-third vessels, or other excipients can be contained in a fourth vessel with a fourth liquid carrier, and so on. When the first liquid carrier is admixed with the second and third liquid carriers, the 3-BP, the biological buffer, and the sugar(s) form a finished 3-BP product in a fourth vessel 608. Such a system allows formulation ingredients to be mixed as needed, which not only extends the potency of the 3-BP, but reduces 3-BP and excipient waste. Due to the reactivity between the 3-BP and the excipient, the 3-BP generally has a lower chemical stability in the finished liquid dosage form compared to the 3-BP prior to admixing.

In some examples, a 3-BP formulation can include a glycolysis inhibitor, one nonlimiting example of which can include 2-deoxglucose (2DOG). The 3-BP formulation can include the glycolysis inhibitor in any effective amount. In the various dosage forms described above, the glycolysis inhibitor can be included with 3-BP, any of the excipients, or in a separate vessel. Provided it does not react with the biological buffer, the glycolysis inhibitor can be admixed therein.

In addition to the above components, the 3-BP compositions described herein can further comprise a halo monocarboxylate compound that is separate from the cellular energy inhibitor. In cases where the halo monocarboxylate compound can function to inhibit glycolysis and/or mitochondria function, the halo monocarboxylate can be considered a second cellular energy inhibitor. In one embodiment, the halo monocarboxylate compound can be a halo two-carbon monocarboxylate compound. The halo two-carbon monocarboxylate compound can be selected from, without limitation, 2-fluoroacetate, 2-chloroacetate, 2-bromoacetate, 2-iodoacetate, and the like, including combinations thereof. In one embodiment, the halo two-carbon monocarboxylate compound can be 2-bromoacetate. In one example, the composition can comprise the halo two-carbon monocarboxylate compound in a concentration from about 0.01 mM to about 5.0 mM. In another example, the composition can comprise a halo two-carbon monocarboxylate compound in a concentration from about 0.1 mM to about 0.5 mM.

Additionally, a halo monocarboxylate compound can be a halo three-carbon monocarboxylate compound. In one embodiment, the halo three-carbon monocarboxylate compound can be selected from, without limitation, 3-fluorolactate, 3-chlorolactate, 3-bromolactate, 3-iodolactate, and the like, including combinations thereof. In another example, the composition can include the halo three-carbon monocarboxylate compound in a concentration from about 0.5 mM to about 250 mM. In one embodiment, the composition can comprise the halo three-carbon monocarboxylate compound in a concentration from about 10 mM to about 50 mM. In the various dosage forms described above in FIGS. 4-7, the halo monocarboxylate compound can be admixed with the 3-BP or be present in a separate layer or in any layer described above, provided the halo monocarboxylate compound is reactively isolated in the storage form.

In some examples, the 3-BP formulations described herein can further comprise a mitochondrial inhibitor in addition to the cellular energy inhibitor. The mitochondrial inhibitor can be selected from, without limitation, oligomycin, efrapeptin, aurovertin, and the like, including combinations thereof. In another example, the composition can include the mitochondrial inhibitor in a concentration from about 0.001 mM to about 5.0 mM. In one example, the composition can include the mitochondrial inhibitor in a concentration from about 0.01 mM to about 0.5 mM. In the various dosage forms described above, the mitochondrial inhibitor can be included with 3-BP, any of the excipients, or in a separate vessel.

In some examples, the present 3-BP formulations can comprise antifungal agents, antibiotics, glycolysis inhibitors, inhibitors of mitochondria, sugars, and biological buffers, without limitation. Examples of such agents include, but are not limited to, amphotericin B, efrapeptin, doxorubicin, (2DOG), analogs of 2DOG, d-lactic acid, dichloroacetic acid (or salt form of dichloroacetate), oligomycin, analogs of oligomycin, glycerol, inositol, sorbitol, glycol, erythritol, threitol, arabitol, xylitol, ribitol, mannitol, dulcitol, iditol, isomalt, maltitol, lactitol, polyglycitol, sodium phosphate, sodium citrate, sodium acetate, sodium carbonate, sodium bicarbonate, sodium pyruvate, sodium lactate, oxaloacetate, isocitrate, aconitate, succinate, fumarate, malate, diluted saline solutions with varying concentrations of NaCl, and water. In addition to the sodium ion that accompanies these biological buffers, calcium and potassium cations can also accompany the biological buffers. Various active agents of the composition can include a cellular energy inhibitor, a glycolysis inhibitor, a mitochondria inhibitor, a halo monocarboxylate compound, an antifungal agent, an antibiotic agent, and the like. In the various dosage forms described above, any of the above ingredients can be included with 3-BP, any of the excipients, or in a separate vessel.

In some examples, the 3-BP compositions described herein can further include a hexokinase inhibitor. In the various dosage forms described above, the hexokinase inhibitor can be included with 3-BP, any of the excipients, or in a separate vessel.

As used herein, “hexokinase 1” or “hexokinase 1 isozyme” refers to any isoforms of hexokinase 1 and its naturally known variants, including those provided in SEQ ID NO: 1, SEQ ID NO: 2, SEQ ID NO: 3, and SEQ ID NO: 4, as follows:

| (SEQ ID NO: 1) | |
|---|---|
| 1 | MIAAQLLAYY FTELKDDQVK KIDKYLYAMR LSDETLIDIM TRFRKEMKNG LSRDFNPTAT |
| 61 | VKMLPTFVRS IPDGSEKGDF IALDLGGSSF RILRVQVNHE KNQNVHMESE VYDTPENIVH |
| 121 | GSGSQLFDHV AECLGDFMEK RKIKDKKLPV GFTFSFPCQQ SKIDEAILIT WTKRFKASGV |
| 181 | EGADVVKLLN KAIKKRGDYD ANIVAVVNDT VGTMMTCGYD DQHCEVGLII GTGTNACYME |
| 241 | ELRHIDLVEG DEGRMCINTE WGAFGDDGSL EDIRTEFDRE IDRGSLNPGK QLFEKMVSGM |
| 301 | YLGELVRLIL VKMAKEGLLF EGRITPELLT RGKFNISDVS AIEKNKEGLH NAKEILTRLG |
| 361 | VEPSDDDCVS VQHVCTIVSF RSANLVAATL GAILNRLRDN KGTPRLRTTV GVDGSLYKTH |
| 421 | PQYSRRFHKT LRRLVPDSDV RFLLSESGSG KGAAMVTAVA YRLAEQHRQI EETLAHFHLT |
| 481 | KDMLLEVKKR MRAEMELGLR KQTHNNAVVK MLPSFVRREP DGTENGDFLA LDLGGTNFRV |
| 541 | LLVKIRSGKK RTVEMHNKIY AIPIEIMQGT GEELFDHIVS CISDFLDYMG IKGPRMPLGF |
| 601 | TFSFPCQQTS LDAGILITWT KGFKATDCVG HDVVTLLRDA IKRREEFDLD VVAVVNDTVG |
| 661 | TMMTCAYEEP TCEVGLIVGT GSNACYMEEM KNVEMVEGDQ GQMCINMEWG AFGDNGCLDD |
| 721 | IRTHYDRLVD EYSLNAGKQR YEKMISGMYL GEIVRNILED FTKKGFLFRG QISETLKTRG |
| 781 | IFETKFLSQI ESDRLALLQV RAILQQLGLN STCDDSILVK TVCGVVSRRA AQLCGAGMAA |
| 841 | VVDKIRENRG LDRLNVTVGV DGTLYKLHPH FSRIMHQTVK ELSPKCNVSF LLSEDGSGKG |
| 901 | AALITAVGVR LRTEASS |
| (SEQ ID NO: 2) | |
| 1 | MDCEHSLSLP CRGAEAWEIG IDKYLYAMRL SDETLIDIMT RFRKEMKNGL SRDFNPTATV |
| 61 | KMLPTFVRSI PDGSEKGDFI ALDLGGSSFR ILRVQVNHEK NQNVHMESEV YDTPENIVHG |
| 121 | SGSQLFDHVA ECLGDFMEKR KIKDKKLPVG FTFSFPCQQS KIDEAILITW TKRFKASGVE |
| 181 | GADVVKLLNK AIKKRGDYDA NIVAVVNDTV GTMMTCGYDD QHCEVGLIIG TGTNACYMEE |
| 241 | LRHIDLVEGD EGRMCINTEW GAFGDDGSLE DIRTEFDREI DRGSLNPGKQ LFEKMVSGMY |
| 301 | LGELVRLILV KMAKEGLLFE GRITPELLTR GKFNTSDVSA IEKNKEGLHN AKEILTRLGV |
| 361 | EPSDDDCVSV QHVCTIVSFR SANLVAATLG AILNRLRDNK GTPRLRTTVG VDGSLYKTHP |
| 421 | QYSRRFHKTL RRLVPDSDVR FLLSESGSGK GAAMVTAVAY RLAEQHRQIE ETLAHFHLTK |
| 481 | DMLLEVKKRM RAEMELGLRK QTHNNAVVKM LPSFVRRTPD GTENGDFLAL DLGGTNFRVL |
| 541 | LVKIRSGKKR TVEMHNKIYA IPIEIMQGTG EELFDHIVSC ISDFLDYMGI KGPRMPLGFT |
| 601 | FSFPCQQTSL DAGILITWTK GFKATDCVGH DVVTLLRDAI KRREEFDLDV VAVVNDTVGT |
| 661 | MMTCAYEEPT CEVGLIVGTG SNACYMEEMK NVEMVEGDQG QMCINMEWGA FGDNGCLDDI |
| 721 | RTHYDRLVDE YSLNAGKQRY EKMISGMYLG EIVRNILIDF TKKGFLFRGQ ISETLKTRGI |
| 781 | FETKFLSQIE SDRLALLQVR AILQQLGINS TCDDSILVKT VCGVVSRRAA QLCGAGMAAV |
| 841 | VDKIRENRGL DRLNVTVGVD GTLYKLHPHF SRIMHQTVKE LSPKCNVSFL LSEDGSGKGA |
| 901 | ALITAVGVRL RTEASS |
| (SEQ ID NO: 3) | |
| 1 | MGQICQRESA TAAEKPKLHL LAESEIDKYL YAMRLSDETL IDIMTRFRKE MKNGLSRDFN |
| 61 | PTATVKMLPT FVRSIPDGSE KGDFIALDLG GSSFRILRVQ VNHEKNQNVH MESEVYDTPE |
| 121 | NIVHGSGSQL FDHVAECLGD FMEKRKIKDK KLPVGFTFSF PCQQSKIDEA ILITWTKRFK |
| 181 | ASGVEGADVV KLLNKAIKKR GDYDANIVAV VNDTVGTMMT CGYDDQHCEV GLIIGTGTNA |
| 241 | CYMEELRHID LVEGDEGRMC INTEWGAFGD DGSLEDIRTE FDREIDRGSL NPGKQLFEKM |
| 301 | VSGMYLGELV RLILVKMAKE GLLFEGRITP ELLTRGKFNT SDVSAIEKNK EGLHNAKEIL |
| 361 | TRLGVEPSDD DCVSVQHVCT IVSFRSANLV AATLGAILNR LRDNKGTPRL RTTVGVDGSL |
| 421 | YKTHPQYSRR FHKTLRRLVP DSDVRFLLSE SGSGKGAAMV TAVAYRLAEQ HRQIEETLAH |
| 481 | FHLTKDMLLE VKKRMRAEME LGLRKQTHNN AVVKMLPSFV RRTPDGTENG DFLALDLGGT |
| 541 | NFRVLLVKIR SGKKRTVEMH NKIYAIPIEI MQGTGEELFD HIVSCISDFL DYMGIKGPRM |
| 601 | PLGFTFSFPC QQTSLDAGIL ITWTKGFKAT DCVGHDVVTL LRDAIKRREE FDLDVVAVVN |
| 661 | DTVGTMMTCA YEEPTCEVGL IVGIGSNACY MEEMKNVEMV EGDQGQMCIN MEWGAFGDNG |
| 721 | CLDDIRTHYD RLVDEYSLNA GKQRYEKMIS GMYLGEIVRN ILIDFTKKGF LFRGQISETL |
| 781 | KTRGIFETKF LSQIESDRLA LLQVRAILQQ LGLNSTCDDS ILVKTVCGVV SRRAAQLCGA |
| 841 | GMAAVVDKIR ENRGLDRLNV TVGVDGTLYK LHPHFSRIMH QTVKELSPKC NVSFLLSEDG |
| 901 | SGKGAALITA VGVRLRTEAS S |
| (SEQ ID NO: 4) | |
| 1 | MAKRALRDFI DKYLYAMRLS DETLIDIMTR FRKEMKNGLS RDFNPTATVK MLPTFVRSIP |
| 61 | DGSEKGDFIA LDLGGSSFRI LRVQVNHEKN QNVHMESEVY DTPENIVHGS GSQLFDHVAE |
| 121 | CLGDFMEKRK IKDKKLPVGF TFSFPCQQSK IDEAILITWT KRFKASGVEG ADVVKLLNKA |
| 181 | IKKRGDYDAN IVAVVNDTVG TMMTCGYDDQ HCEVGLIIGT GTNACYMEEL RHIDLVEGDE |
| 241 | GRMCINTEWG AFGDDGSLED IRTEFDREID RGSLNPGKQL FEKMVSGMYL GELVRLILVK |
| 301 | MAKEGLLFEG RITPELLTRG KFNTSDVSAI EKNKEGLHNA KEILTRLGVE PSDDDCVSVQ |
| 361 | HVCTIVSFRS ANLVAATLGA ILNRLRDNKG TPRLRTTVGV DGSLYKTHPQ YSRRFHKTLR |
| 421 | RLVPDSDVRF LLSESGSGKG AAMVTAVAYR LAEQHRQIEE TLAHFHLTKD MLLEVKKRMR |
| 481 | AEMELGLRKQ THNNAVVKML PSFVRRTPDG TENGDFLALD LGGTNFRVLL VKIRSGKKRT |
| 541 | VEMHNKIYAI PIEIMQGTGE ELFDHIVSCI SDFLDYMGIK GPRMPLGFTF SFPCQQTSLD |
| 601 | AGILITWTKG FKATDCVGHD VVTLLRDAIK RREEFDLDVV AVVNDTVGTM MTCAYEEPTC |
| 661 | EVGLIVGTGS NACYMEEMKN VEMVEGDQGQ MCINMEWGAF GDNGCLDDIR THYDRLVDEY |
| 721 | SLNAGKQRYE KMISGMYLGE IVRNILIDFT KKGFLFRGQI SETLKTRGIF ETKFLSQIES |
| 781 | DRLALLQVRA ILQQLGLNST CDDSILVKTV CGVVSRRAAQ LCGAGMAAVV DKIRENRGLD |
| 841 | RLNVTVGVDG TLYKLHPHFS RIMHQTVKEL SPKCNVSFLL SEDGSGKGAA LITAVGVRLR |
| 901 | TEASS |

As used herein, “hexokinase 2” or “hexokinase 2 isozyme” refers to any isoforms of hexokinase 2 and its naturally known variants, including that provided in SEQ ID NO: 5 as follows:

| (SEQ ID NO: 5) | |
|---|---|
| 1 | MIASHLLAYF FTELNHDQVQ KVDQYLYHMR LSDETLLEIS KRFRKEMEKG LGATTHPTAA |
| 61 | VKMLPTFVRS TPDGTEHGEF LALDLGGTNF RVLWVKVTDN GLQKVEMENQ IYAIPEDIMR |
| 121 | GSGTQLFDHI AECLANFMDK LQIKDKKLPL GFTFSFPCHQ TKLDESFLVS WTKGFKSSGV |
| 181 | EGRDVVALIR KAIQRRGDFD IDIVAVVNDT VGTMMTCGYD DHNCEIGLIV GTGSNACYME |
| 241 | EMRHIDMVEG DEGRMCINME WGAFGDDGSL NDIRTEFDQE IDMGSLNPGK QLFEKMISGM |
| 301 | YMGELVRLIL VKMAKEELLF GGKLSPELLN TGREETKDIS DIEGEKDGIR KAREVLMRLG |
| 361 | LDPTQEDCVA THRICQIVST RSASLCAATL AAVLQRIKEN KGEERLRSTI GVDGSVYKKH |
| 421 | PHFAKRLHKT VRRLVPGCDV RFLRSEDGSG KGAAMVTAVA YRLADQHRAR QKTLEHLQLS |
| 481 | HDQLLEVKRR MKVEMERGLS KETHASAPVK MLPTYVCATP DGTEKGDFLA LDLGGTNFRV |
| 541 | LLVRVRNGKW GGVEMHNKIY AIPQEVMHGT GDELFDHIVQ CIADFLEYMG MKGVSLPLGF |
| 601 | TFSFPCQQNS LDESILLKWT KGFKASGCEG EDVVTLLKEA IHRREEFDLD VVAVVNDTVG |
| 661 | TMMTCGFEDP HCEVGLIVGT GSNACYMEEM RNVELVEGEE GRMCVNMEWG AFGDNGCLDD |
| 721 | FRTEFDVAVD ELSLNPGKQR FEKMISGMYL GEIVRNILID FTKRGLLFRG RISERLKTRG |
| 781 | IFETKFLSQI ESDCLALLQV RAILQHLGLE STCDDSIIVK EVCTVVARRA AQLCGAGMAA |
| 841 | VVDRIRENRG LDALKVTVGV DGTLYKLHPH FAKVMHETVK DLAPKCDVSF LQSEDGSGKG |
| 901 | AALITAVACR IREAGQR |

In some examples, the 3-BP formulations described herein can further comprise a hexokinase inhibitor. The hexokinase inhibitor can be any molecule that inhibits hexokinase 1, hexokinase 2, and/or any isozyme thereof (collectively referred to herein as “hexokinase”).

As has been described, a major source of ATP production occurs in mitochondria in normal cells. However, ATP production from glycolysis is significantly upregulated in cancer cells. One reason for this upregulation is due to hexokinase molecules binding to, and forming complexes with, mitochondrial voltage dependent anion channels (VDACs) at ATP synthasomes, thus forming so called “ATP synthasome mega complexes.” The formation of such ATP synthasome mega complexes can immortalize the cancer cell, thus allowing the continued use of the cell's energy production processes for cancer growth. A hexokinase inhibitor, therefore, can thus block hexokinase from binding to the VADCs or displace hexokinase molecules from the VADCs of already formed ATP synthasome mega complexes.

In one example, a hexokinase inhibitor can be up to 25 amino acid units from the N-terminal region of Hexokinase 2 isozyme or Hexokinase 1 isozyme. In another example, the hexokinase inhibitor can be an amino acid sequence of 5 to 20 amino acid units, where the 5 to 20 amino acid sequence is present in the first 25 amino acid unit region beginning from the N-terminal end of hexokinase 1 isozyme or hexokinase 2 isozyme. In one example, the 5 to 20 amino acid sequence can be any 5-20 amino acid sequence present in the first 25 amino acid unit region of the N-terminus of Hexokinase 1 1 or Hexokinase 2. Such amino acid sequences can displace cellular bound hexokinase or competitively bind to voltage dependent anion channels (VDAC), thus preventing initial hexokinase binding.

In other examples, a hexokinase inhibitor can include antibodies against a portion of HK1 or HK2, such as, for example, the N-terminal region of either molecule. In one specific example, a hexokinase inhibitor can be an amino acid sequence, such as SEQ ID NO: 6, corresponding to the first 25 amino acids from the N-terminus end of hexokinase 1 (isoform1) having a sequence as follows:

| (SEQ ID NO: 6) | |
|---|---|
| 1 | MIAAQLLAYY FTELKDDQVK KIDKY |

In another example, a hexokinase inhibitor can be an amino acid sequence as in SEQ ID NO: 7, corresponding to the first 25 amino acids from the N-terminus end of hexokinase 1 (isoform 2) having a sequence as follows:

| (SEQ ID NO: 7) | |
|---|---|
| 1 | MDCEHSLSLP CRGAEAWEIG IDKYL |

In yet another example, a hexokinase inhibitor can be an amino acid sequence as in SEQ ID NO: 8, corresponding to the first 25 amino acids from the N-terminus end of hexokinase 1 (isoform 3) having a sequence as follows:

| (SEQ ID NO: 8) | |
|---|---|
| 1 | MGQICQRESA TAAEKPKLHL LAESE |

In still another example, a hexokinase inhibitor can be an amino acid sequence as in SEQ ID NO: 9, corresponding to the first 25 amino acids from the N-terminus end of hexokinase 1 (isoform 4) having a sequence as follows:

| (SEQ ID NO: 9) | |
|---|---|
| 1 | MAKRALRDFI DKYLYAMRLS DETLI |

In yet another example, a hexokinase inhibitor can be an amino acid sequence as in SEQ ID NO: 10, corresponding to the first 25 amino acids from the N-terminus end of hexokinase 2 having a sequence as follows:

| (SEQ ID NO: 10) | |
|---|---|
| 1 | MIASHLLAYF FTELNHDQVQ KVDQY |

Additional hexokinase inhibitors can be those as disclosed in U.S. Pat. No. 5,854,067 (to Newgard et al, issued Dec. 29, 1998) and/or U.S. Pat. No. 5,891,717 (to Newgard et al., issued Apr. 6, 1999), both of which are incorporated by reference in their entireties. Additional hexokinase inhibitors that can be used in the present formulations include those disclosed in U.S. Pat. Nos. 6,670,330; 6,218,435; 5,824,665; 5,652,273; and 5,643,883; and U.S. patent application publication Nos. 20030072814; 20020077300; and 20020035071; each of the foregoing patent publications and patent application is incorporated herein by reference, in their entireties.

In some examples, the 3-BP compositions described herein can further comprise various ingredients recited below. In the various dosage forms described above in FIGS. 4-7, any of these various ingredients can be admixed with the 3-BP, provided they are nonreactive therewith, or be present in a separate layer or in any layer described above, provided the ingredient(s) is/are reactively isolated in the storage form.

In one embodiment, the present compositions can include less biologically active amino acids as compared to their isomers to facilitate cancer cell starvation. In one aspect, the less biologically active amino acid can be a D-amino acid. However, if the L-amino acid is less biologically active than the D-form, the L-amino acid can be used.

In one embodiment, the present compositions can include inhibitors for DNA replication; inhibitors for DNA binding; and/or inhibitors for DNA transcription. In another embodiment, the present compositions can include inhibitors for cell cycle, growth and/or proliferation. In yet another embodiment, the present compositions can include inhibitors for signal transduction pathways. In yet another embodiment, the present compositions can include inhibitors for angiogenesis. In yet another embodiment, the present compositions can include small RNAs that interfere with normal gene control including antisense RNA, micro RNA, small hairpin RNA, short hairpin RNA, small interfering RNA, and the like. In yet another embodiment, the present compositions can include vitamin C; nutritional supplements including vitamins, CoQ10, flavonoids, free fatty acid, alpha lipoic acid, acai, gogi, mango, pomergrante, L-carnitine, selenium; etc.

### EXAMPLES

The following examples pertain to specific embodiments and point out specific features, elements, or steps that can be used or otherwise combined in achieving such embodiments.

In one example, a system of compartmentalized ingredients for a liquid pharmaceutical formulation can include:
a first vessel including an active pharmaceutical ingredient (API) in a first liquid carrier; and
a second vessel including an excipient in a second liquid carrier that is chemically reactive with the API, wherein admixing the first liquid carrier with the second liquid carrier creates a finished liquid dosage form.

In another example of a system of compartmentalized ingredients for a liquid pharmaceutical formulation, the API in the finished liquid dosage form has a lower chemical stability compared to the API prior to admixing.

In another example, a system of compartmentalized ingredients for a liquid pharmaceutical formulation can further include an additional excipient in the first liquid carrier with the API.

In another example of a system of compartmentalized ingredients for a liquid pharmaceutical formulation, the additional excipient is substantially non-chemically reactive with the API.

In one example, a system of compartmentalized ingredients for a liquid pharmaceutical formulation can include:
a first vessel including a cellular energy inhibitor according to formula I in a first liquid carrier, wherein Halo includes a member selected from the group consisting of fluoro, chloro-, bromo-, and iodo-; and
a second vessel including an excipient in a second liquid carrier, wherein admixing the first liquid carrier with the second liquid carrier creates a finished liquid 3-halopyruvate dosage form

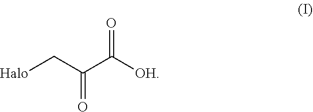

In another example of a system of compartmentalized ingredients for a liquid pharmaceutical formulation, Halo is bromo- and the cellular energy inhibitor is 3-bromopyruvate (3-BP) according to formula (II)

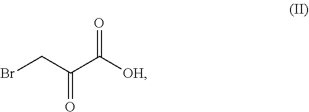

and the finished liquid 3-halopyruvate dosage form is a finished liquid 3-BP dosage form.

In another example of a system of compartmentalized ingredients for a liquid pharmaceutical formulation, the excipient in the second carrier is chemically reactive with the 3-BP.

In another example of a system of compartmentalized ingredients for a liquid pharmaceutical formulation, the 3-BP in the finished liquid 3-BP dosage form has a lower chemical stability compared to the 3-BP prior to admixing.

In another example of a system of compartmentalized ingredients for a liquid pharmaceutical formulation, the excipient in the second liquid carrier includes at least one sugar, which stabilizes the 3-BP in the finished liquid 3-BP dosage form by substantially preventing the 3-BP from hydrolyzing.

In another example of a system of compartmentalized ingredients for a liquid pharmaceutical formulation, the excipient in the second liquid carrier includes a biological buffer that is present in an amount sufficient to at least partially deacidify the 3-BP in the finished liquid 3-BP dosage form and to at least partially neutralize metabolic by-products of the 3-BP in the finished liquid 3-BP dosage form.

In another example of a system of compartmentalized ingredients for a liquid pharmaceutical formulation, the excipient in the second liquid carrier includes:
a biological buffer that is present in an amount sufficient to at least partially deacidify the 3-BP in the finished liquid 3-BP dosage form and to at least partially neutralize metabolic by-products of the 3-BP in the finished liquid 3-BP dosage form; and
at least one sugar, which stabilizes the 3-BP in the finished liquid 3-BP dosage form by substantially preventing the 3-BP from hydrolyzing.

In another example, a system of compartmentalized ingredients for a liquid pharmaceutical formulation can include an additional excipient in the first liquid carrier.

In another example of a system of compartmentalized ingredients for a liquid pharmaceutical formulation, the additional excipient in the first liquid carrier includes a biological buffer that is present in an amount sufficient to at least partially deacidify the 3-BP in the finished liquid 3-BP dosage form and to at least partially neutralize metabolic by-products of the 3-BP in the finished liquid 3-BP dosage form.

In another example of a system of compartmentalized ingredients for a liquid pharmaceutical formulation, the additional excipient in the first liquid carrier includes at least one sugar, which stabilizes the 3-BP by substantially preventing the 3-BP from hydrolyzing.

In another example of a system of compartmentalized ingredients for a liquid pharmaceutical formulation, the additional excipient in the first liquid carrier includes a biological buffer that is present in an amount sufficient to at least partially deacidify the 3-BP in the finished liquid 3-BP dosage form and to at least partially neutralize metabolic by-products of the 3-BP in the finished liquid 3-BP dosage form.

In another example, a system of compartmentalized ingredients for a liquid pharmaceutical formulation can include a third vessel including a further excipient in a third liquid carrier, wherein admixing the first liquid carrier with the second liquid carrier and the third liquid carrier creates a finished liquid 3-BP dosage form.

In another example of a system of compartmentalized ingredients for a liquid pharmaceutical formulation, the excipient in the second liquid carrier includes a biological buffer that is present in an amount sufficient to at least partially deacidify the 3-BP in the finished liquid 3-BP dosage form and to at least partially neutralize metabolic by-products of the 3-BP in the finished liquid 3-BP dosage form and the further excipient in the third liquid carrier includes at least one sugar, which stabilizes the 3-BP by substantially preventing the 3-BP from hydrolyzing.

In another example of a system of compartmentalized ingredients for a liquid pharmaceutical formulation, the at least one sugar can be selected from gluconic acid, glucuronic acid, mannitol, erythritol, isomalt, lactitol, maltitol, sorbitol, xylitol, dulcitol, ribitol, inositol, myo inositol, glycerol, ethylene glycol, threitol, arabitol, galactitol, fucitol, iditol, volemitol, maltotriitol, maltotetraitol, polyglycitol, or a combination thereof.

In another example of a system of compartmentalized ingredients for a liquid pharmaceutical formulation, the at least one sugar can be a five-carbon sugar.

In another example of a system of compartmentalized ingredients for a liquid pharmaceutical formulation, the at least one sugar can be at least two five-carbon sugars.

In another example of a system of compartmentalized ingredients for a liquid pharmaceutical formulation, the composition can include a second sugar selected from mannitol, erytritol, isomalt, lactitol, maltitol, sorbitol, xyolitol, dulcitol, ribitol, inositol, myo inositol, sorbitol, and combinations thereof.

In another example, a system of compartmentalized ingredients for a liquid pharmaceutical formulation can include a second sugar and a third sugar independently selected from mannitol, erytritol, isomalt, lactitol, maltitol, sorbitol, xyolitol, dulcitol, ribitol, inositol, myo inositol, sorbitol, and combinations thereof.

In another example of a system of compartmentalized ingredients for a liquid pharmaceutical formulation, the at least one sugar can include glycerol, myo inositol, and sorbitol.

In another example, a system of compartmentalized ingredients for a liquid pharmaceutical formulation can include one or more sugars in a range from about 0.5 wt % to about 50.0 wt % or from about 1.0 wt % to about 25.5 wt %. In yet another example, a composition can include one or more sugars in a range from about 0.2 wt % to about 75.0 wt % or from about 0.5 wt % to about 50.0 wt %. In a further example, a composition can include one or more sugars in a range from about 0.1 wt % to about 25.0 wt %, from about 0.2 wt % to about 10.0 wt %.

In another example, a system of compartmentalized ingredients for a liquid pharmaceutical formulation can include glycerol in a range from about 0.1 wt % to about 5.0 wt % or from about 0.1 wt % to about 3.0 wt %. In other examples, the composition can include inositol in a range from about 0.1 wt % to about 10 wt %, from about 0.1 wt % to about 6 wt %. In further examples, the composition can include sorbitol in a range from about 0.1 wt % to about 40.0 wt % or from about 0.1 wt % to about 30 wt %. In yet further examples, the composition can include mannitol in a range from about 0.1 wt % to about 30 wt % or from about 0.1 wt % to about 10 wt %. Additionally, each of the sugars may be added in a volume up to a maximum solubility of the sugar in the formulation or composition.

In another example, a system of compartmentalized ingredients for a liquid pharmaceutical formulation can include d-lactic acid and epinephrine.

In another example, a system of compartmentalized ingredients for a liquid pharmaceutical formulation can include a glycolysis inhibitor and wherein the glycolysis inhibitor is 2-deoxglucose in a concentration from about 1 mM to about 5 mM.

In another example, a system of compartmentalized ingredients for a liquid pharmaceutical formulation can include the glycolysis inhibitor 2-deoxglucose.

In another example, a system of compartmentalized ingredients for a liquid pharmaceutical formulation can include the 2-deoxglucose in a concentration from about 1 mM to about 5 mM.

In another example of a system of compartmentalized ingredients for a liquid pharmaceutical formulation, the biological buffer is selected from one or more of a citrate buffer, a phosphate buffer, and an acetate buffer.

In another example of a system of compartmentalized ingredients for a liquid pharmaceutical formulation, the biological buffer is selected from one or more of a citrate buffer, a phosphate buffer, and an acetate buffer.

In another example of a system of compartmentalized ingredients for a liquid pharmaceutical formulation, the biological buffer is a citrate buffer.

In another example of a system of compartmentalized ingredients for a liquid pharmaceutical formulation, the biological buffer is a phosphate buffer.

In another example, a system of compartmentalized ingredients for a liquid pharmaceutical formulation can include at least one additive selected from phospholipids; liposomes; nanoparticles; immune system modulators and/or immune system boosters including brown rice extract, muramyl dipeptide including analogues, mushroom extract, bioflavonoids, Vitamin D3-Binding Protein-Derived Macrophage Activating Factor (GcMAF), inhibitors of nagalase, threonine attached to N-acetylgalactosamine, and antibodies against nagalase; L-lactate dehydrogenase; D-lactate dehydrogenase; nicotinamide adenine dinucleotides; inhibitors for DNA replication; inhibitors for DNA binding; inhibitors for DNA transcription; inhibitors for cell cycle, growth and/or proliferation; inhibitors for signal transduction pathways; inhibitors for angiogensis; small RNAs that interfere with normal gene control including antisense RNA, micro RNA, small hairpin RNA, short hairpin RNA, small interfering RNA; vitamin C; nutritional supplements including vitamins, CoQ10, flavonoids, free fatty acid, alpha lipoic acid, acai, gogi, mango, pomergrante, L-carnitine, selenium; a less biologically active amino acid as compared to its isomer; and mixtures thereof.

In another example, a system of compartmentalized ingredients for a liquid pharmaceutical formulation can include a hexokinase inhibitor.

In another example of a system of compartmentalized ingredients for a liquid pharmaceutical formulation, the hexokinase inhibitor inhibits binding of hexokinase 1 and/or hexokinase 2 to VDAC.

In another example of a system of compartmentalized ingredients for a liquid pharmaceutical formulation, the hexokinase inhibitor is an amino acid sequence selected from the group consisting of: SEQ ID NO: 6, SEQ ID NO: 7, SEQ ID NO: 8, SEQ ID NO: 9, and SEQ ID NO. 10.

In another example of a system of compartmentalized ingredients for a liquid pharmaceutical formulation, the composition can include a mitochondrial inhibitor.

In another example of a system of compartmentalized ingredients for a liquid pharmaceutical formulation, the mitochondrial inhibitor is selected from oligomycin, efrapeptin, aurovertin, and mixtures thereof in a concentration from about 0.01 mM to about 0.5 mM.

In another example of a system of compartmentalized ingredients for a liquid pharmaceutical formulation, the mitochondrial inhibitor is in a concentration from about 0.01 mM to about 0.5 mM.

What is claimed is:

1. A system of compartmentalized ingredients for a liquid pharmaceutical formulation, comprising:
a first vessel including a cellular energy inhibitor according to formula I in a first liquid carrier, 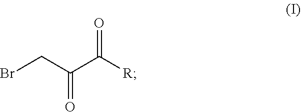
wherein R is selected from one of OR′, N(R″)₂, C(O)R′″, C1-C6 alkyl, C6-C12 aryl, C1-C6 heteroalkyl, C6-C12 heteroaryl, H, or an alkali metal,
where R′ is selected from one of H, an alkali metal, C1-C6 alkyl, C6-C12 aryl or C(O)R′″,
where R″ is selected from one of H, C1-C6 alkyl, or C6-C12 aryl,
and where R′″ is selected from one of H, C1-C20 alkyl or C6-C12 aryl; and
a second vessel including an excipient in a second liquid carrier that is chemically reactive with the cellular energy inhibitor, wherein admixing the first liquid carrier with the second liquid carrier creates a finished liquid dosage form.

2. The formulation of claim 1, wherein the cellular energy inhibitor has a structure according to formula II.

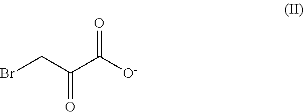

3. The formulation of claim 1, wherein the cellular energy inhibitor has a structure according to formula III.

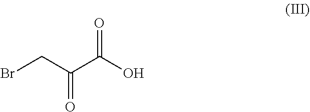

4. The system of claim 1, where in the cellular energy inhibitor in the finished liquid dosage form has a lower chemical stability compared to the cellular energy inhibitor prior to admixing.

5. The system of claim 1, further comprising an additional excipient in the first liquid carrier.

6. The system of claim 5, wherein the additional excipient is substantially non-chemically reactive with the API.

7. The system of claim 1, wherein the excipient in the second liquid carrier includes at least one sugar which stabilizes the cellular energy inhibitor in the finished liquid dosage form by substantially preventing the cellular energy inhibitor from hydrolyzing.

8. The system of claim 7, wherein the at least one sugar is selected from gluconic acid, glucuronic acid, mannitol, erythritol, isomalt, lactitol, maltitol, sorbitol, xylitol, dulcitol, ribitol, inositol, myo inositol, glycerol, ethylene glycol, threitol, arabitol, galactitol, fucitol, iditol, volemitol, maltotriitol, maltotetraitol, polyglycitol, or a combination thereof.

9. The system of claim 7, wherein the at least one sugar is a five-carbon sugar.

10. The system of claim 7, wherein the at least one sugar is at least two five-carbon sugars.

11. The system of claim 7, wherein the second liquid carrier further includes a second sugar selected from mannitol, erytritol, isomalt, lactitol, maltitol, sorbitol, xyolitol, dulcitol, ribitol, inositol, myo inositol, or sorbitol.

12. The system of claim 7, wherein the second liquid carrier further includes a second sugar and a third sugar independently selected from mannitol, erytritol, isomalt, lactitol, maltitol, sorbitol, xyolitol, dulcitol, ribitol, inositol, myo inositol, sorbitol, or a combination thereof.

13. The system of claim 7, wherein the at least one sugar can include glycerol, myo inositol, and sorbitol.

14. The system of claim 7, wherein the at least one sugar is present in a range from about 0.1 wt % to about 25.0 wt %, from about 0.2 wt % to about 10.0 wt %.

15. The system of claim 7, wherein the at least one sugar includes glycerol in a range from about 0.1 wt % to about 5.0 wt % or from about 0.1 wt % to about 3.0 wt %, inositol in a range from about 0.1 wt % to about 10 wt %, from about 0.1 wt % to about 6 wt %, and sorbitol in a range from about 0.1 wt % to about 40.0 wt % or from about 0.1 wt % to about 30 wt %.

16. The system of claim 1, wherein the excipient in the second liquid carrier includes a biological buffer that is present in an amount sufficient to at least partially deacidify the cellular energy inhibitor in the finished liquid dosage form and to at least partially neutralize metabolic by-products of the cellular energy inhibitor in the finished liquid dosage form.

17. The system of claim 1, wherein the excipient in the second liquid carrier includes:
a biological buffer that is present in an amount sufficient to at least partially deacidify the cellular energy inhibitor in the finished liquid dosage form and to at least partially neutralize metabolic by-products of the cellular energy inhibitor in the finished liquid dosage form; and
at least one sugar, which stabilizes the cellular energy inhibitor in the finished liquid dosage form by substantially preventing the cellular energy inhibitor from hydrolyzing.

18. The system of claim 1, further comprising a third vessel including a further excipient in a third liquid carrier, wherein admixing the first liquid carrier with the second liquid carrier and the third liquid carrier creates the finished liquid dosage form.

19. The system of claim 18, wherein the excipient in the second liquid carrier includes a biological buffer that is present in an amount sufficient to at least partially deacidify the cellular energy inhibitor in the finished liquid dosage form and to at least partially neutralize metabolic by-products of the cellular energy inhibitor in the finished liquid dosage form and the further excipient in the third liquid carrier includes at least one sugar, which stabilizes the cellular energy inhibitor by substantially preventing the from hydrolyzing.

20. The system of claim 19, wherein the biological buffer is selected from one or more of a citrate buffer, a phosphate buffer, or an acetate buffer.

21. The system of claim 1, wherein at least one of the first liquid carrier or the second liquid carrier further includes at least one additive selected from phospholipids; liposomes; nanoparticles; immune system modulators and/or immune system boosters including brown rice extract, muramyl dipeptide including analogues, mushroom extract, bioflavonoids, Vitamin D3-Binding Protein-Derived Macrophage Activating Factor (GcMAF), inhibitors of nagalase, threonine attached to N-acetylgalactosamine, and antibodies against nagalase; L-lactate dehydrogenase; D-lactate dehydrogenase; nicotinamide adenine dinucleotides; inhibitors for DNA replication; inhibitors for DNA binding; inhibitors for DNA transcription; inhibitors for cell cycle, growth and/or proliferation; inhibitors for signal transduction pathways; inhibitors for angiogensis; small RNAs that interfere with normal gene control including antisense RNA, micro RNA, small hairpin RNA, short hairpin RNA, small interfering RNA; vitamin C; nutritional supplements including vitamins, CoQ10, flavonoids, free fatty acid, alpha lipoic acid, acai, gogi, mango, pomergrante, L-carnitine, selenium; a less biologically active amino acid as compared to its isomer; and mixtures thereof.

22. The system of claim 1, wherein at least one of the first liquid carrier or the second liquid carrier further includes a hexokinase inhibitor.

23. The system of claim 22, wherein the hexokinase inhibitor inhibits binding of hexokinase 1 and/or hexokinase 2 to VDAC.

24. The system of claim 22, wherein at least one of the first liquid carrier or the second liquid carrier further includes a mitochondrial inhibitor selected from oligomycin, efrapeptin, aurovertin, and mixtures thereof in a concentration from about 0.01 mM to about 0.5 mM.

25. The system of claim 1, wherein at least one of the first liquid carrier or the second liquid carrier further includes 2-deoxglucose in a concentration from about 1 mM to about 5 mM.
